(19) United States
(12) Patent Application Publication
(10) Pub. No.: US 2023/0348900 A1
(43) Pub. Date: Nov. 2, 2023

(54) HIGH-THROUGHPUT METHODS FOR THE DETECTION OF ECTOPIC INTEGRATION OF TRANSFORMING DNA

(71) Applicant: Zymergen Inc., Emeryville, CA (US)

(72) Inventors: Benjamin KNOX, Oakland, CA (US); Johannes Wilhelmus Adrianus VAN DIJK, Alameda, CA (US)

(21) Appl. No.: 18/021,747

(22) PCT Filed: Aug. 19, 2021
(86) PCT No.: PCT/US2021/046653

§ 371 Date: Feb. 16, 2023

Related U.S. Application Data
(60) Provisional application No. 63/068,169, filed on Aug. 20, 2020.

(51) Int. Cl. C12N 15/10

(52) U.S. Cl. CPC C12N 15/1082

(57) ABSTRACT

The present disclosure provides compositions and methods for detection of ectopic integration of a DNA fragment. The disclosure further provides a method for determining the quality of a genomic transformation.

### CROSS-REFERENCE TO RELATED APPLICATIONS

This application claims priority to U.S. Provisional Application No. 63/068,169, filed on Aug. 20, 2020, which is incorporated by reference herein in its entirety.

### FIELD

The present disclosure generally describes compositions and methods for detection of ectopic integration of a DNA fragment. The disclosure further provides a method for determining the quality of genomic transformation.

### BACKGROUND

Ectopic integration occurs when transforming DNA integrates into the genome of an organism at an off-target location via non-homologous end joining (NHEJ). In some organisms, ectopic integration occurs with a frequency comparable to the frequency of homologous recombination at the correct target. Ectopic integration can have significant and oftentimes deleterious consequences for individual genomes as a result of its effect on genome structure or through the unintentional alteration of an off-target gene. Ectopic integration may lead to genomic deletions, inversions, translocations, and other rearrangements.

The location of an ectopic integration is unpredictable, complicating the detection of such integrations. Moreover, current techniques, e.g., Southern Blot or Whole Genome Sequencing (WGS) to detect ectopic integration are time consuming and inefficient. Thus, there exists a need in the art for new high-throughput methods of detecting ectopic integration.

### SUMMARY

The present disclosure solves the problems in the art by providing compositions and methods for detecting ectopic integration. The present disclosure provides novel methods and compositions for detecting ectopic integration. The compositions of the disclosure comprise a genetic element comprising a first tail, a second tail, a first homology arm, a second homology arm, a primer that binds the first tail, and a primer that binds the second tail. The disclosure further provides methods for using these compositions to detect ectopic integration. Moreover, the disclosure provides methods of determining the quality of a genomic transformation in a cell population comprising detecting ectopic integration of a DNA fragment and assigning the cell population a quality score based on the presence or absence of ectopic integration.

In some embodiments, provided herein is a method for detecting ectopic integration of a genetic element comprising:
(a) transforming a cell with a genetic element, comprising a first tail, a second tail, a first homology arm, and a second homology arm;
wherein the first tail is distal to the first homology arm and the second tail is distal to the second homology arm; and
(b) detecting ectopic integration of the genetic element by determining if a first tail or second tail is present in the genome of the cell;
wherein the presence of a first tail or second tail indicates ectopic integration.

In some embodiments, provided herein is a composition for detecting ectopic integration, comprising:
(a) a genetic element, wherein the genetic element comprises a first tail, a second tail, a first homology arm, and a second homology arm;
wherein the first tail is distal to the first homology arm and the second tail is distal to the second homology arm;
(b) a primer that binds to the first tail; and
(c) a primer that binds to the second tail.

In some embodiments, provided herein is a composition for detecting ectopic integration in a cell, comprising:
(a) a genetic element, wherein the genetic element comprises a first tail, a second tail, a first homology arm, and a second homology arm;
wherein the first tail is distal to the first homology arm and the second tail is distal to the second homology arm;
(b) a primer that binds to the first tail, and
(c) a primer that binds to the second tail.

In some embodiments, the genetic element of the composition or method described herein comprises two nucleic acids, wherein from 5′ to 3′, the first nucleic acid comprises the first tail, a first homology arm, and the second tail and wherein from 5′ to 3′, the second nucleic acid comprises a third tail, the second homology arm and a fourth tail.

In some embodiments, the genetic element of the composition or method described herein comprises two nucleic acids, wherein from 5′ to 3′, the first nucleic acid comprises the first tail, a first homology arm, and the second tail and wherein from 5′ to 3′, the second nucleic acid comprises a third tail, the second homology arm and a fourth tail, wherein the first tail and third tail have identical nucleic acid sequences.

In some embodiments, the genetic element of the composition or method described herein comprises two nucleic acids, wherein from 5′ to 3′, the first nucleic acid comprises the first tail, a first homology arm, and the second tail and wherein from 5′ to 3′, the second nucleic acid comprises a third tail, the second homology arm and a fourth tail, wherein the second tail and fourth tail have identical nucleic acid sequences.

In some embodiments, the genetic element of the composition or method described herein comprises two nucleic acids, wherein from 5′ to 3′, the first nucleic acid comprises the first tail, a first homology arm, and the second tail and wherein from 5′ to 3′, the second nucleic acid comprises a third tail, the second homology arm and a fourth tail, wherein the first tail and third tail have identical nucleic acid sequences., and wherein the second tail and fourth tail have identical nucleic acid sequences.

In some embodiments, the presence of a third or a fourth tail in the genome of the cell indicates ectopic integration.

In some embodiments, the genetic element of the composition or method described herein comprises two nucleic acids, wherein 3′ of the first homology arm, the first nucleic acid comprises a fragment of a selectable marker gene.

In some embodiments, the genetic element of the composition or method described herein comprises two nucleic acids, wherein 5′ of the second homology arm, the second nucleic acid comprises a fragment of a selectable marker gene.

In some embodiments, the genetic element of the composition or method described herein comprises two nucleic acids, wherein 3′ of the first homology arm the first nucleic acid comprises a payload.

In some embodiments, the genetic element of the composition or method described herein comprises two nucleic acids, wherein 5′ of the second homology arm the second nucleic acid comprises a payload.

In some embodiments, the genetic element of the composition or method described herein comprises two nucleic acids, wherein the first nucleic acid and the second nucleic acid comprise a payload.

In some embodiments, the genetic element of the composition or method described herein comprises a nucleic acid, and wherein from 5′ to 3′, the nucleic acid comprises the first tail, the first homology arm, the second homology arm, and the second tail.

In some embodiments, the genetic element of the composition or method described herein comprises a nucleic acid, and wherein from 5′ to 3′, the nucleic acid comprises the first tail, the first homology arm, the second homology arm, and the second tail, and wherein the nucleic acid comprises a selectable marker gene.

In some embodiments, the genetic element of the composition or method described herein comprises a nucleic acid, and wherein from 5′ to 3′, the nucleic acid comprises the first tail, the first homology arm, the second homology arm, and the second tail, and wherein the nucleic acid comprises a payload.

In some embodiments, the genetic element of the composition or method described herein comprises a nucleic acid, and wherein from 5′ to 3′, the nucleic acid comprises the first tail, the first homology arm, the second homology arm, and the second tail, and wherein the nucleic acid comprises a selectable marker gene, wherein the selectable marker gene is located between the first homology arm and the second homology arm.

In some embodiments, the genetic element of the composition or method described herein comprises a nucleic acid, and wherein from 5′ to 3′, the nucleic acid comprises the first tail, the first homology arm, the second homology arm, and the second tail, and wherein the nucleic acid comprises a payload, wherein the payload is located between the first homology arm and the second homology arm.

In some embodiments, the cell of the composition or methods described herein is a prokaryotic cell.

In some embodiments, the cell of the composition or methods described herein is a eukaryotic cell.

In some embodiments, the cell of the composition or methods described herein is a fungal cell.

In some embodiments, the first tail, second tail, third tail, fourth tail, and combinations thereof, of the compositions and methods described herein comprises a unique nucleic acid sequence that is not found in the genome of the transformed cell.

In some embodiments, the nucleic acid sequence of the first tail, second tail, third tail, fourth tail, and combinations thereof, of the compositions and methods described herein comprises a primer binding site.

In some embodiments, the nucleic acid sequence of the first tail, second tail, third tail, fourth tail, and combinations thereof, of the compositions and methods described herein comprises a GC content of between about 40% and about 60%.

In some embodiments, the nucleic acid sequence of the first tail, second tail, third tail, fourth tail, and combinations thereof, of the compositions and methods described herein exhibits low self-complementarity.

In some embodiments, the first tail, second tail, third tail, fourth tail, and combinations thereof of the methods described herein are detected using a primer.

In some embodiments, the first tail, second tail, third tail, fourth tail, and combinations thereof of the methods described herein are detected using a primer, wherein the primer comprises a fluorophore or a radioisotope.

In some embodiments, the methods comprise confirming ectopic integration of a DNA fragment using Southern Blotting or whole-genome sequencing.

In some embodiments, provided herein is a method of determining the quality of genomic transformation in a cell population that is transformed with a genetic element comprising a first tail, a second tail, a first homology arm, a second homology arm, and a DNA fragment, comprising:
(a) detecting ectopic integration of a DNA fragment by determining if a first tail or second tail is present in the genome of the cell, wherein the presence of a first tail or second tail indicates ectopic integration; and
(b) assigning the cell population a quality score, wherein a cell population that contains an ectopic integration has a lower quality score than a cell population without ectopic integration.

In some embodiments, the method comprises assigning a cell population with on-target integration a higher quality score than a cell population without on-target integration.

In some embodiments, the method comprises assigning a monoclonal cell population a higher quality score than a polyclonal cell population.

### BRIEF DESCRIPTION OF THE DRAWINGS

FIGS. 1A-C show example cloning vectors showing the locations of the first tail, second tail, first homology arm (L-HOM), and second homology arm (R-HOM). FIG. 1A shows a standard cloning backbone containing a first tail, a second tail, and a payload cloning site. FIG. 1B shows a standard cloning backbone containing a first tail, a second tail, and a payload (“targeted gene editing sequence”). FIG. 1C shows one vector containing a first tail, a first homology arm (L-HOM), the 5′ portion of the selectable marker gene pyr4, and a second tail and a second vector containing the first tail, a second homology arm (R-HOM), the 3′ portion of the selectable marker gene pyr4, and a second tail. Polymerase chain reaction is utilized to generate two linear nucleic acids, one which contains a first tail, a first homology arm (L-HOM), the 5′ portion of the selectable marker gene pyr4, and a second tail and a second which contains a first tail, the second homology arm (R-HOM), the 3′ portion of the selectable marker gene pyr4, and a second tail.

FIG. 2 shows that homologous recombination using the two nucleic acids of FIG. 1C results in replacement of genE (a genomic target) with pyr4 and exclusion of the tail sequences.

FIG. 3 shows that non-homologous end joining using the two nucleic acids of FIG. 1C results off-target incorporation of pyr4 and integration of tail sequences. The tail sequences can be detected using PCR. The arrows indicate primer binding sites on the tail sequences.

FIG. 4 shows an exemplary method of incorporating ectopic integration detection with a high-throughput, next generation sequencing platform to assess the quality of genomic editing.

FIG. 5 shows exemplary genetic elements containing one or two nucleic acids. Genetic elements may be used to delete, edit, or insert nucleic sequences in the genomic target.

FIG. 6 shows a design strategy for the generation of tail sequences. Barcode sequences or a random sequence generator may be used to generate potential tail sequences. Software such as BLAST is used to align tail sequences to a target genome. A homology threshold is employed to identify and prioritize tail sequences based on low homology to genome of interest.

### DETAILED DESCRIPTION

### 1. Definitions

The term “a” or “an” refers to one or more of that entity, i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% (i.e., within 10%, 9%, 8%, 7%, 6%, 5%, 4%, 3%, 2%, 1%, or less) above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

Herein, the terms “comprise”, or variations such as “comprises” or “comprising”, will be understood to imply the inclusion of a stated element or integer or group of elements or integers but not the exclusion of any other element or integer or group of elements or integers.

A “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (the aforementioned Bacteria and Archaea) is that they have membrane-bound organelles, especially the nucleus, which contains the genetic material, and is enclosed by the nuclear envelope.

“Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

As used herein, the term “fungus” or “fungi” refers in general to any organism from Kingdom Fungi. Historical taxonomic classification of fungi has been according to morphological presentation. Beginning in the mid-1800's, it was became recognized that some fungi have a pleomorphic life cycle, and that different nomenclature designations were being used for different forms of the same fungus. In 1981, the Sydney Congress of the International Mycological Association laid out rules for the naming of fungi according to their status as anamorph, teleomorph, or holomorph (Taylor, 2011). With the development of genomic sequencing, it became evident that taxonomic classification based on molecular phylogenetics did not align with morphological-based nomenclature (Shenoy, 2007). As a result, in 2011 the International Botanical Congress adopted a resolution approving the International Code of Nomenclature for Algae, Fungi, and Plants (Melbourne Code) (2012), with the stated outcome of designating “One Fungus=One Name” (Hawksworth, 2012). However, systematics experts have not aligned on common nomenclature for all fungi, nor are all existing databases and information resources inclusive of updated taxonomies. As such, many fungi referenced herein may be described by their anamorph form but it is understood that based on identical genomic sequencing, any pleomorphic state of that fungus may be considered to be the same organism. For example, the genus Alternaria is the anamorph form of the teleomorph genus Lewia (Kwasna 2003), ergo both would be understood to be the same organism with the same DNA sequence. For example, it is understood that the genus Acremonium is also reported in the literature as genus Sarocladium as well as genus Tilachilidium (Summerbell, 2011). For example, the genus Cladosporium is an anamorph of the teleomorph genus Davidiella (Bensch, 2012), and is understood to describe the same organism. In some cases, fungal genera have been reassigned due to various reasons, and it is understood that such nomenclature reassignments are within the scope of any claimed genus. For example, certain species of the genus Mierodiplodia have been described in the literature as belonging to genus Paraconiothyrium (Crous and Groenveld, 2006).

As used herein, “selectable marker” is a nucleic acid segment that allows one to select for a molecule (e.g., a replicon) or a cell that contains it, often under particular conditions. These markers can encode an activity, such as, but not limited to, production of RNA, peptide, or protein, or can provide a binding site for RNA, peptides, proteins, inorganic and organic compounds or compositions and the like. Examples of selectable markers include but are not limited to: (1) nucleic acid segments that encode products which provide resistance against otherwise toxic compounds (e.g., antibiotics); (2) nucleic acid segments that encode products which are otherwise lacking in the recipient cell (e.g., tRNA genes, auxotrophic markers); (3) nucleic acid segments that encode products which suppress the activity of a gene product; (4) nucleic acid segments that encode products which can be readily identified (e.g., phenotypic markers such as p-galactosidase, green fluorescent protein (GFP), yellow fluorescent protein (YFP), cyan fluorescent protein (CFP), and cell surface proteins); (5) nucleic acid segments that encode products that bind other products which are otherwise detrimental to cell survival and/or function; (6) nucleic acid segments that encode nucleic acids that otherwise inhibit the activity of any of the nucleic acid segments resulting in a visible or selectable phenotype (e.g., antisense oligonucleotides); (7) nucleic acid segments that encode products that bind other products that modify a substrate (e.g. restriction endonucleases); (8) nucleic acid segments that can be used to isolate or identify a desired molecule (e.g. specific protein binding sites); (9) nucleic acid segments that encode a specific nucleotide sequence which can be otherwise non-functional (e.g., for PCR amplification of subpopulations of molecules); and (10) nucleic acid segments, which when absent, directly or indirectly confer resistance or sensitivity to particular compounds.

As used herein, “counterselectable marker” or a “counterselection marker” is a nucleic acid segment that eliminates or inhibits growth of a host organism upon selection. In some embodiments, the counterselectable markers of the present disclosure render the cells sensitive to one or more chemicals/growth conditions/genetic backgrounds. In some embodiments, the counterselectable markers of the present disclosure are toxic genes. In some embodiments, the counterselectable markers are expressed by inducible promoters.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably. In some embodiments, nucleotides contain ribose, deoxyribose, or analogs thereof, for example, 2′-O-methyl, 2′-O-allyl, 2′-fluoro or 2′-Azidoribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogues such as methyl riboside. In some embodiments, one or more phosphodiester bonds of a nucleic acid may be replaced with alternative groups. Alternative groups include, but are not limited to P (O) S (“thioate”), P (S) S (“dithioate”), (O) NR₂ (“amidate”), P (O)R, P (O) OR′, CO or CH₂ (“formacetal”), in which each R or R′ is independently H or substituted or unsubstituted alkyl (1-20 C), optionally an ether—(—O—)-bond, aryl, alkenyl, cycloalkyl, cycloalkenyl or araldyl. Not all bonds in a polynucleotide must be identical. The foregoing description is applicable to all of the nucleic acids referred to herein, including RNA and DNA.

In some embodiments, a nucleotide or nucleic acid is labeled. In some embodiments, a nucleotide is labeled according to methods known in the art. In some embodiments, the nucleotide is labeled with a dye and/or a detectable moiety such as a specific binding pair member (e.g. biotin-avidin). Labeled dNTP or rNTP may also be indirect be marked by its attachment to, for example, a component to which a marker is/may be attached. A dNTP or rNTP may comprise a molecular moiety (for example, an amino group or hydrazide group) to which a label is attached. Non-limiting examples of labels include fluorescent dyes (e.g., fluorescein isothiocyanate, Texas Red, rhodamine, green fluorescent protein and the like), radioisotopes (e.g. ³H, ³⁵S, ³²P, ³³P, ¹²⁵I or ¹⁴C), enzymes (eg LacZ, horseradish peroxidase, alkaline phosphatase), digoxigenin, and colorimetric labels such as colloidal gold or colored glass or plastic beads (e.g., polystyrene, polypropylene, latex, etc.). Various anti-ligands and ligands may be used (as labels themselves or as a label attachment agent).

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “promoter” refers to a DNA sequence capable of controlling the expression of a coding sequence or functional RNA. The promoter sequence may consist of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter.

The term “competent cell” refers to a cell which has the ability to take up and replicate an exogenous nucleic acid.

As used herein, an “extra-chromosomally replicating plasmid” is an autonomously replicating vector that exists as an extra-chromosomal entity. The replication of an extra-chromosomally replicating plasmid is independent of chromosomal replication.

The term “ribonucleoprotein” as used herein refers to a RNA sequence associated with a protein. The association of RNA and protein may be effected by any suitable means, including, for example, protein-nucleic acid interactions. In other words the term “ribonucleoprotein” as used herein may refer to a RNA-protein complex.

The term “endonuclease” or “nuclease” refers to any wild-type or mutant enzyme that has the ability to catalyze the hydrolysis (cleavage) of bonds between nucleic acids within a DNA or RNA molecule.

The term “recombinase” generally refers to an enzyme that catalyzes recombination.

The term “transform” refers to the introduction of a molecule, such as a polynucleotide, into a competent cell.

The term “ectopic integration” means the insertion of a nucleic acid into the genome of a microorganism at a non-targeted site or at a site other than its usual chromosomal locus, i.e., random integration.

The term “fragment” refers to a portion of a nucleic acid (e.g. a promoter, a gene, an exon, or an intron) or a protein. In some embodiments, the fragment is about 0.1%, about 0.2%, about 0.3%/0, about 0.4%/0, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of the nucleobases or amino acids of a nucleic acid or protein. In some embodiments, the fragment is at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 30, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39°,%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% of the nucleobases or amino acids of a nucleic acid or protein.

A “high-throughput (HTP)” method of genomic engineering may involve the utilization of at least one piece of automated equipment (e.g. a liquid handler or plate handler machine) to carry out at least one step of said method.

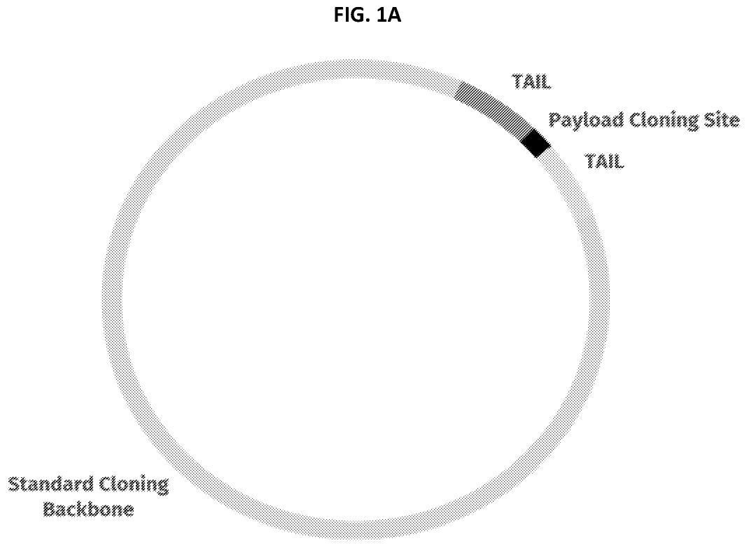

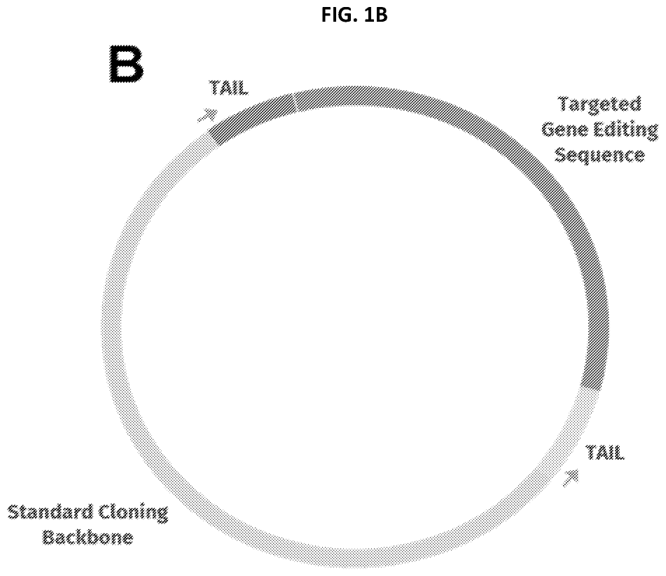

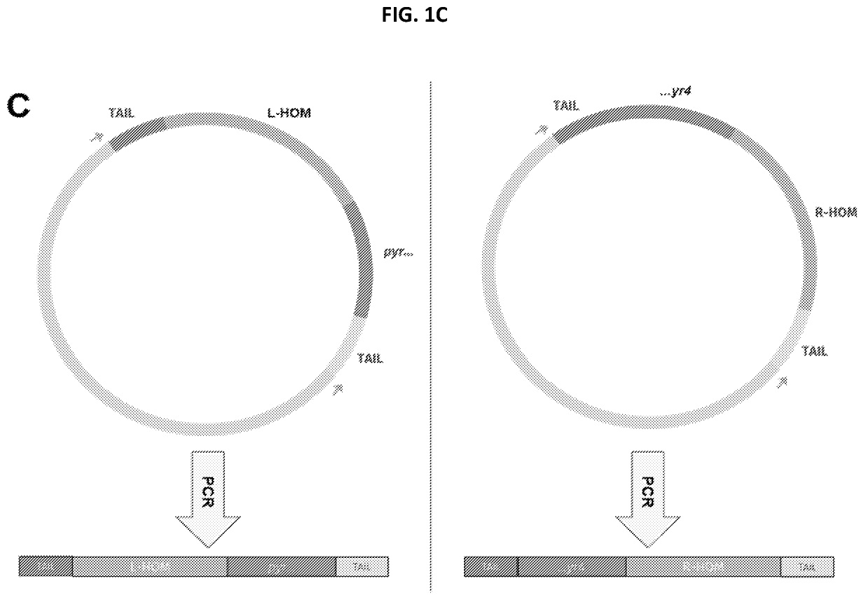

The term “distal” as it refers to a tail of a nucleic acid described herein means that the tail is located on the 5′ or the 3′ end of a nucleic acid relative to a reference region of the nucleic acid (e.g., a homology arm, selectable marker gene, payload, or a combination thereof). In embodiments, a nucleic acid comprises two tails, a first tail and a second tail that are distal to a reference region, wherein the first tail is located 5′ of the reference region and the second tail is located 3′ to the reference region of the nucleic acid. For example, the linear nucleic acids of FIG. 1C each contain two tails that are distal to a homology arm (L-HOM or R-HOM).

### II. Compositions for Gene Editing

Described herein are compositions for gene editing which enable detection of ectopic integration (also called “off-target integration”). The compositions described herein can be used with any of the methods described in Sections m or IV of the present disclosure.

Ectopic integration occurs when transforming DNA (e.g., a genetic element as described herein) integrates into the genome of an organism at an off-target location via non-homologous end joining (NHEJ). In contrast, desired on-target integration in which transforming DNA (e.g., a genetic element as described herein) integrates at a predetermined locus occurs via homologous recombination (HR).

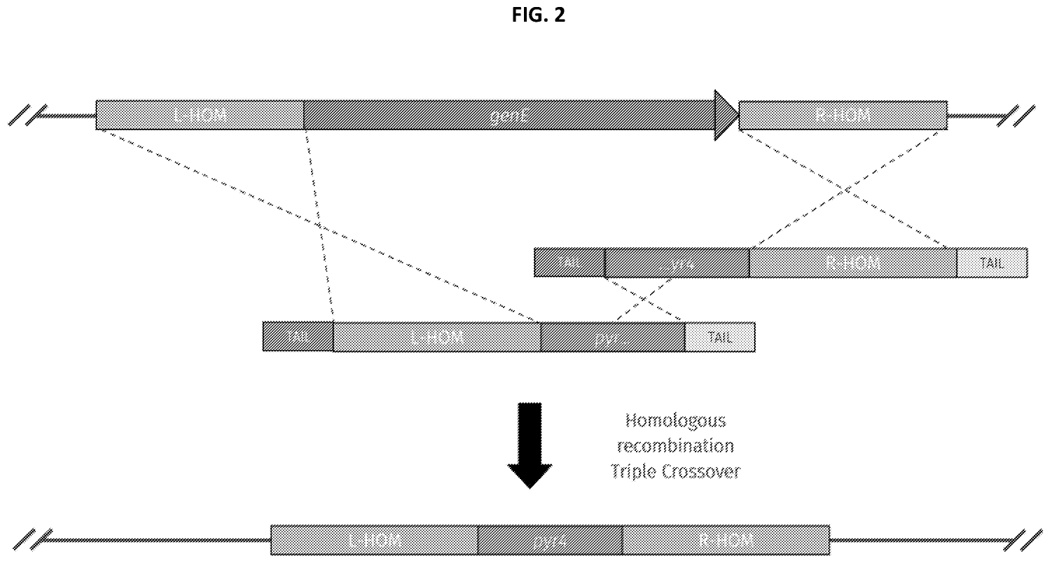

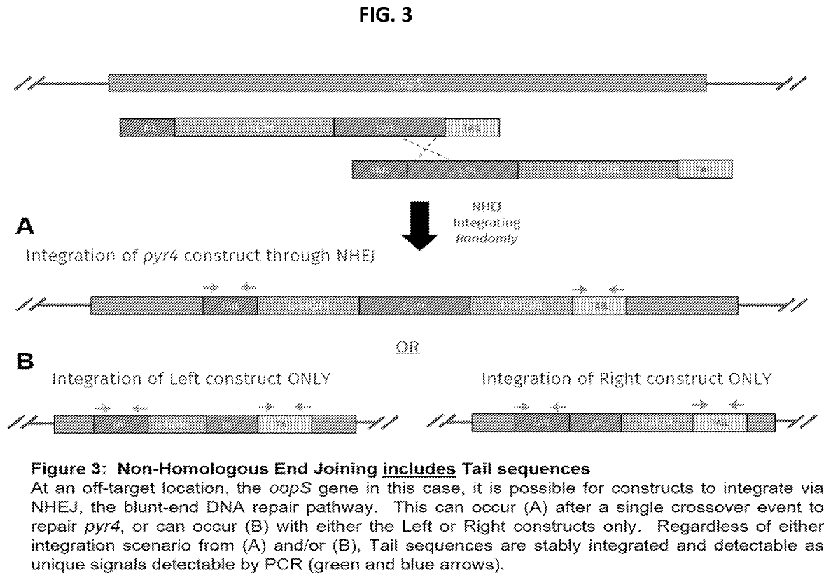

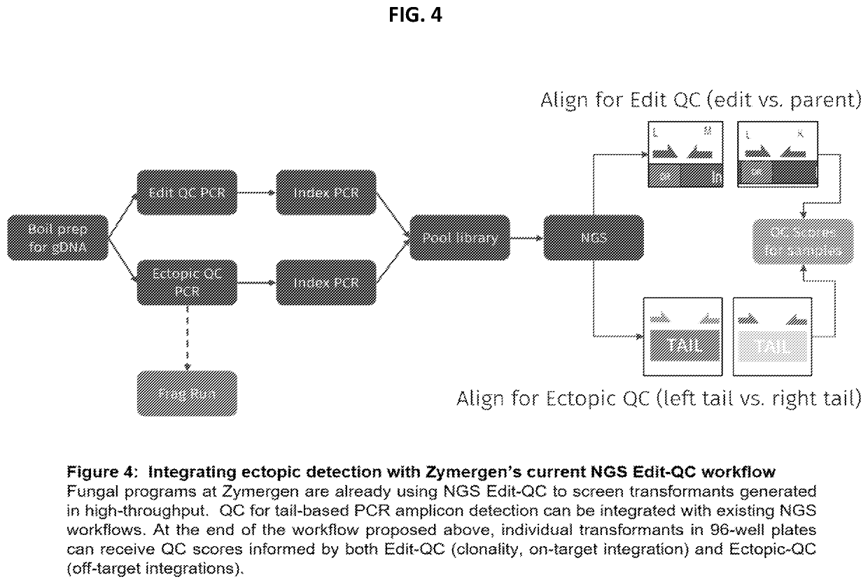

The compositions provided herein utilize nucleic acids called tails to discriminate between ectopic integration and on-target integration of a genetic element. Tails are contained within each genetic element described herein. A “tail” comprises a primer binding site that is retained during non-homologous end joining and removed during targeted homologous recombination. During HR, tails are removed as a consequence of their location on the transforming linear DNA distal to any sequences that participate in recombination including L-HOM, R-HOM, and split-marker elements (FIG. 2). As genetic elements crossover during HR there is a joining of DNA molecules at homologous sequences, and when linear transforming DNA is used as in the present disclosure, elements distal to homologous sequences get removed while sequences between double crossover events, such as payloads, get integrated into the genome. During NHEJ, linear DNA molecules are directly ligated together regardless of homologous sequences which, as outlined herein, would incorporate linear transforming DNAs in their entirety including Tail sequences (FIG. 3).

The compositions for detecting ectopic integration of a genetic element, comprise:
(a) A genetic element, wherein the genetic element comprises a first tail, a second tail, a first homology arm, and a second homology arm; wherein the first tail is distal to the first homology arm and the second tail is distal to the second homology arm;
(b) A primer pair that binds within the first tail; and
(c) A primer pair that binds within the second tail.

The compositions provided herein are used to detect ectopic integration of one or more payloads, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 payloads, in the genome of a cell.

In some embodiments, the compositions described herein are used in any of the methods of the disclosure described in Sections III and IV of this disclosure.

### A. Genetic Element

In some embodiments, the compositions described herein comprise a genetic element. As used herein, a “genetic element” is a nucleic acid comprising a first tail, a second tail, a first homology arm, and a second homology arm. In some embodiments, the genetic element is a deoxyribonucleic acid (DNA). In some embodiments, the genetic element is a ribonucleic acid (RNA). In some embodiments, the genetic element is linear. In some embodiments, the genetic element is circular.

In some embodiments, the genetic element is double stranded. In some embodiments, the genetic element is single stranded. In some embodiments, the genetic element is partially double stranded. In some embodiments, a partially double stranded genetic element comprises single stranded overhangs, at the 5′ and/or 3′ of the genetic element. In some embodiments, the single stranded overhangs comprise between about 1 and 100 nucleotides, for example, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides.

In some embodiments, the genetic element is prepared using polymerase chain reaction. In some embodiments, the genetic element is prepared using a restriction digest. In some embodiments, the genetic element is synthesized.

In some embodiments, the compositions comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 genetic elements.

In some embodiments, a genetic element comprises a fragment of a nucleic acid, for example, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of a nucleic acid. In some embodiments, the fragment comprises about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800, about 810, about 820, about 830, about 840, about 850, about 860, about 870, about 880, about 890, about 900, about 910, about 920, about 930, about 940, about 950, about 960, about 970, about 980, about 990, or about 1000 nucleotides of a nucleic acid sequence.

In some embodiments, the genetic element encodes for a fragment of a protein. In some embodiments, the genetic element encodes about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of a protein. In some embodiments, the fragment of a protein comprises about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800, about 810, about 820, about 830, about 840, about 850, about 860, about 870, about 880, about 890, about 900, about 910, about 920, about 930, about 940, about 950, about 960, about 970, about 980, about 990, or about 1000 amino acids of a protein sequence.

In some embodiments, a genetic element comprises one nucleic acid. In some embodiments, a genetic element comprising one nucleic acid comprises from 5′ to 3′ a first tail, a first homology arm, a second homology arm, and a second tail. In some embodiments, the genetic element comprises a payload, selectable marker gene, or both. In some embodiments, a payload or selectable marker gene is located between the first homology arm and the second homology arm.

In some embodiments, a genetic element comprises two nucleic acids, the “first nucleic acid” and the “second nucleic acid.” In some embodiments, from 5′ to 3′, the first nucleic acid comprises a first tail and a first homology arm. In some embodiments, from 5′ to 3′, the second nucleic acid comprises a second homology arm and a second tail. In some embodiments, from 5′ to 3′, the first nucleic acid comprises a first tail, a first homology arm, and a second tail. In some embodiments, from 5′ to 3′, the second nucleic acid comprises a first tail, a second homology arm and a second tail. In some embodiments, from 5′ to 3′, the second nucleic acid comprises a third tail, a second homology arm and a fourth tail. In some embodiments, the first nucleic acid, second nucleic acid, or both comprise a payload, or fragment thereof. In some embodiments, the first nucleic acid, second nucleic acid, or both comprise a selectable marker gene, or fragment thereof. In some embodiments, the first tail of the first and second nucleic acids is identical. In some embodiments, the second tail of the first and second nucleic acids is identical. In some embodiments, the first tail of the first and second nucleic acids is not identical. In some embodiments, the second tail of the first and second nucleic acids is not identical.

### Tails

In some embodiments, the genetic elements described herein comprise a first tail and a second tail. As used herein, a “tail” is a nucleic acid component of the genetic element taught herein located distal to a homology arm or split-marker element of a genetic element taught herein, wherein the presence of the tail sequence in genomic DNA of a transformed organism indicates ectopic integration (e.g. off-target integration) of the genetic element. The term “tail” and “tail sequence” are used interchangeable, as well as the term “tail marker sequence” or “tail tag sequence,” all of which denote the aforementioned nucleic acid sequence component of the taught genetic elements that is utilized to detect off-target/ectopic integration of the genetic elements. When designing tail nucleic acid sequences, the following structural considerations, inter alia, are taken into account: (1) sequence length; (2) sequence identity to the genome of a transformed organism; (3) sequence identity to the payload of the genetic element; (4) percentage of GC content; (5) the number of polynucleotide stretches; (6) self-complementarity between portions of a tail sequence; and (7) primer binding near the 5′ and 3′ end of a tail.

In some embodiments, the tail comprises between about 1 and about 200 nucleic acids, for example, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, or about 200 nucleotides. In some embodiments, the tail is greater than 200 nucleotides. In some embodiments, the tail comprises a gene. In some embodiments, the tail encodes for a protein. In some embodiments, the tail encodes for a protein. In some embodiments, the tail encodes for an antibiotic resistance gene.

In some embodiments, a tail has low homology to a sequence of interest. Exemplary sequences of interests include a genome of any of the organisms described herein, a selectable marker gene, or a payload. In some embodiments, a tail has less than about 10%, less than about 9%, less than about 8%, less than about 7%, less than about 6%, less than about 5%, less than about 4%, less than about 3%, less than about 2%, less than about 1%, less than about 0.9%, less than about 0.8%, less than about 0.7%, less than about 0.6%, less than about 0.5%, less than about 0.4%, less than about 0.3%, less than about 0.2%, less than about 0.1% identity to a sequence of interest. Percentage identity can be calculated using the tools CLUSTAL OMEGA, EMBOSS Needle or Basic Local Alignment Search Tool (BLAST), which are available online. The following default parameters may be used for EMBOSS NEEDLE Pairwise alignment: Protein Weight Matrix=BLOSUM62; Gap Open=10. Gap Extension=0.5. In some embodiments, a first tail or second tail comprises a unique nucleic acid sequence that is not found in the genome of a target cell.

In some embodiments, a tail comprises between about 40% and about 70% GC content (the percentage of bases in the tail which are guanine or cytosine), for example, about 40%, about 45%, about 50%, about 55%, about 60%, about 65%, or about 70% GC content. In some embodiments, a tail comprises between about 40% and about 60% GC content. In some embodiments, the tail has a GC content of about 50%.

In some embodiments, a tail comprises one or more primer binding sites. In some embodiments, the primer binding site is located within the first 20% of nucleic acids starting from the 5′ end of the tail. For example, if a tail comprises 200 nucleic acids, a primer binding site may be located within 40 nucleotides from the 5′ end of the tail. In some embodiments, the primer binding site is located within the last 20% of nucleic acids. For example, if a tail comprises 200 nucleic acids, a primer binding site may be located within about 40 nucleotides from the 3′ and/or 5′ end of the tail.

In some embodiments, a tail comprises a low number of mononucleotide repeats.

In some embodiments, a tail comprises a low self-complementarity score. A self-complementarity score is the likelihood that a tail will bind to itself. In some embodiments, routine DNA analysis tools are used to calculate a complementarity score. In some embodiments, the self-complementarity score is detected using Primer3Plus.

In some embodiments, a genetic element comprising two nucleic acids comprises a first tail on the first nucleic acid and a second tail on the second nucleic acid. In some embodiments, the first nucleic acid comprises a first tail that is located 5′ of the first homology arm. In some embodiments, the second nucleic acid comprises a second tail that is located 3′ of the second homology arm.

In some embodiments, a genetic element comprising two nucleic acids comprises a first tail and a second tail on a first nucleic acid and a first tail and a second tail on the second nucleic acid. In some embodiments, the first nucleic acid comprises a first tail that is located 5′ of the first homology arm and a second tail that is located 3′ of the first homology arm. In some embodiments, the second nucleic acid comprises a first tail that is located 5′ of the second homology arm and a second tail that is located 3′ of the second homology arm.

In some embodiments, a genetic element comprising two nucleic acids comprises a first tail and a second tail on a first nucleic acid and a third tail and a fourth tail on the second nucleic acid. In some embodiments, the first nucleic acid comprises a first tail that is located 5′ of the first homology arm and a second tail that is located 3′ of the first homology arm. In some embodiments, the second nucleic acid comprises a third tail that is located 5′ of the second homology arm and a fourth tail that is located 3′ of the second homology arm. In some embodiments, the first tail and third tail comprise identical nucleic acid sequences. In some embodiments, the second tail and fourth tail comprise identical nucleic acid sequences. In some embodiments, the first tail and third tail comprise identical nucleic acid sequences, and the second tail and fourth tail comprise identical nucleic acid sequences.

In some embodiments, a first tail is located 5′ of the first homology arm and the second tail is located 3′ of the second homology arm of a genetic element comprising one nucleic acid.

In some embodiments, a first tail is distal to the first homology arm and a second tail is distal to the second homology arm of a genetic element.

### Homology Arms

In some embodiments, a genetic element described herein contains a homology arm. A homology arm is a nucleic acid sequence that exhibits homology to a fragment of the genome of a cell. In some embodiments, the genetic elements described herein contain a first homology arm and a second homology arm.

In some embodiments, a homology arm comprises between about 1 and about 10,000 nucleotides, for example, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800, about 810, about 820, about 830, about 840, about 850, about 860, about 870, about 880, about 890, about 900, about 910, about 920, about 930, about 940, about 950, about 960, about 970, about 980, about 990, about 1000, about 1050, about 1100, about 1150, about 1200, about 1250, about 1300, about 1350, about 1400, about 1450, about 1500, about 1550, about 1600, about 1650, about 1700, about 1750, about 1800, about 1850, about 1900, about 2000, about 2100, about 2200, about 2300, about 2400, about 2500, about 2600, about 2700, about 2800, about 2900, about 3000, about 3100, about 3200, about 3300, about 3400, about 3500, about 3600, about 3700, about 3800, about 3900, about 4000, about 4100, about 4200, about 4300, about 4400, about 4500, about 4600, about 4700, about 4800, about 4900, about 5000, about 5100, about 5200, about 5300, about 5400, about 5500, about 5600, about 5700, about 5800, about 5900, about 6000, about 6100, about 6200, about 6300, about 6400, about 6500, about 6600, about 6700, about 6800, about 6900, about 7000, about 7100, about 7200, about 7300, about 7400, about 7500, about 7600, about 7700, about 7800, about 7900, about 8000, about 8100, about 8200, about 8300, about 8400, about 8500, about 8600, about 8700, about 8800, about 8900, about 9000, about 9100, about 9200, about 9300, about 9400, about 9500, about 9600, about 9700, about 9800, about 9900, or about 10000 nucleotides.

In some embodiments, a genetic element comprising two nucleic acids comprises a first homology arm on a first nucleic acid and a second homology arm on the second nucleic acid. In some embodiments, the first nucleic acid comprises a first homology arm that is located 3′ of the first tail. In some embodiments, the second nucleic acid comprises a second homology arm that is located 5′ of the second tail.

In some embodiments, a first homology arm is located 3′ of a first tail and a second homology arm is located 5′ of the second tail of a genetic element comprising one nucleic acid.

### Selectable Marker Gene

In some embodiments, a genetic element comprises a selectable marker gene. In some embodiments, the selectable marker gene is an antibiotic resistance gene, for example, a chloramphenicol resistance gene, an ampicillin resistance gene, a tetracycline resistance gene, a Zeocin resistance gene, a spectinomycin resistance gene and a Km (Kanamycin resistance gene), tetA (tetracycline resistance gene), G418 (neomycin resistance gene), van (vancomycin resistance gene), methicillin (methicillin resistance gene), penicillin (penicillin resistance gene), oxacillin (oxacillin resistance gene), erythromycin (erythromycin resistance gene), linezolid (linezolid resistance gene), puromycin (puromycin resistance gene) or a hygromycin (hygromycin resistance gene). In some embodiments, the selectable marker gene is selected from pyrG, hph, nat, amdS, nptII, niaD, and argB.

In some embodiments, a genetic element comprising two nucleic acids comprises a fragment of a selectable marker gene on the first nucleic acid and a fragment of a selectable marker gene on the second nucleic acid. In some embodiments, the first nucleic acid comprises a fragment of a selectable marker gene that is located 3′ of the first homology arm. In some embodiments, the second nucleic acid comprises a fragment of a selectable marker gene that is located 5′ of the second homology arm.

In some embodiments, a selectable marker gene is located between the first homology arm and the second homology arm of a genetic element comprising one nucleic acid.

In some embodiments, the selectable marker gene is flanked by short segments of identical sequence (e.g., a direct repeat). In some embodiments, the direct repeat is a nucleic acid sequence that is identical to an existing genomic sequence or to a sequence within the genetic element. Direct repeats flank a region of DNA slated for looping-out and deletion. Once inserted, cells containing the loop out construct or constructs can be counter selected for deletion of the region of DNA.

### Payload

In some embodiments, the genetic elements described herein comprise a payload. In some embodiments, a “payload” comprises a nucleic acid sequence to be inserted into the genome of a cell (e.g., a target cell) at a specific location (e.g., a genomic target). In some embodiments, a genetic element comprises between about 1 and about 200 payloads, for example, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, or about 200 payloads.

In some embodiments, the payload is selected from the group consisting of: a nucleic acid sequence, a gene of interest, a gene variant, a genetic edit, a single nucleotide polymorphism, a genetic regulatory sequence, a promoter, a non-coding nucleic acid sequence, a terminator, or any combination thereof. In some embodiments, the payload is a biosynthetic gene cluster or a portion of a biosynthetic gene cluster. A biosynthetic gene cluster is an organized group of genes responsible for the production of one or more compounds.

In some embodiments, the payload is a gene (referred to interchangeably as a “gene of interest.” In some embodiments, the gene is exogenous to a target cell. In some embodiments, the gene is endogenous to the target cell. In some embodiments, the gene of interest encodes for an enzyme, a transporter, a regulatory protein, a substrate-binding protein, a surface-active protein, or a structural protein. In some embodiments, the product of the “gene of interest” can be located intracellularly or extracellularly. In some embodiments, the product of a gene of interest is expressed as a secreted protein. In some embodiments, the gene of interest comprises a mutation compared to the wild-type gene of interest. The mutation can be an insertion, deletion, substitution, or single-nucleotide polymorphism. In some embodiments, the gene comprises a genetic regulatory or control element (e.g. a promoter or a terminator). In some embodiments, the gene is flanked by a genetic regulatory or control element (e.g. a promoter or a terminator).

In some embodiments, the payload is a promoter or a terminator sequence. The promoter sequence and/or terminator sequence can be endogenous or heterologous relative to the variant strain and/or the parental strain. Promoter sequences can be operably linked to the 5′ termini of the sequences to be expressed. A variety of known fungal promoters are likely to be functional in the disclosed cells, for example, the promoter sequences of C1 endoglucanases, the 55 kDa cellobiohydrolase (CBHl), glyceraldehyde-3-phosphate dehydrogenase A, C. lucknowense GARG 27K and the 30 kDa xylanase (XyIF) promoters from Chrysosporium, as well as the Aspergillus promoters described in, e.g. U.S. Pat. Nos. 4,935,349; 5,198,345; 5,252,726; 5,705,358; and 5,965,384; and PCX application WO 93/07277. Terminator sequences can be operably linked to the 3′ termini of the sequences to be expressed. A variety of known fungal terminators are likely to be functional in the disclosed host strains. Examples are the A. nidulans trpC terminator, A. niger alpha-glucosidase terminator, A. niger glucoamylase terminator, Mucor miehei earboxyl protease terminator (see U.S. Pat. No. 5,578,463), Chrysosporium terminator sequences, e.g. the EG6 terminator, and the Trichoderma reesei cellobiohydrolase terminator.

In some embodiments, a payload comprises a genetic edit. A genetic edit can be an insertion of a payload into the genome of a cell, substitution of a portion of genomic DNA within a target cell with a payload, or generation of a single-nucleotide polymorphism within a target cell.

In some embodiments, the payload is a single nucleotide polymorphism.

In some embodiments, the payload is a genetic regulatory sequence.

In some embodiments, the payload is a non-coding nucleic acid sequence.

In some embodiments, the payload of interest is linear, single-stranded DNA. In some embodiments, the payload is linear, double-stranded DNA. In some embodiments, the payload comprises one or more sticky ends. As used herein, a “sticky end” is a region of unpaired nucleotides at the end of a DNA double helix. In some embodiments, a payload is linear.

In some embodiments, a payload is a vector. In some embodiments, a vector comprises a payload. In some embodiments, the vector is an integrative vector. An integrative vector becomes integrated into the genome and replicated together with the chromosome(s) into which it has been integrated. An integrative vector may integrate at random or at a predetermined genomic target site of a target cell.

In some embodiments, a payload is a selectable marker gene. A payload may comprise an selectable marker gene described herein.

In some embodiments, a first nucleic acid and second nucleic each have a payload.

### B. Primers

In some embodiments, the compositions comprise a primer. In some embodiments, a first primer binds to a first tail. In some embodiments, a second primer binds to a second tail. In some embodiments, a third primer binds to a third tail. In some embodiments, a fourth primer binds to a fourth tail. In some embodiments, a first primer binds to a first tail and a second primer binds to a second tail. In some embodiments, a first primer binds to a first tail, a second primer binds to a second tail, a third primer binds to a third tail, and a fourth primer binds to a fourth tail.

In some embodiments, the compositions comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 primers.

In some embodiments, a primer comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleic acids.

In some embodiments, a primer may be complementary to any fragment of a genetic element. In some embodiments, a primer may be complementary to genomic DNA in a target cell.

In some embodiments, a primer is complementary to a tail. In some embodiments, a primer is complementary to a first tail, a second tail, a third tail, a fourth tail, or any combination thereof. In some embodiments, a primer comprises between about 40% and about 70% GC content (the percentage of bases in the tail which are guanine or cytosine), for example, about 40%, about 45%, about 50%, about 55%, about 60%, about 65%, or about 70% GC content. In some embodiments, the primer has a GC content of about 50%.

In some embodiments, a primer comprises a low number of polynucleotide stretches.

In some embodiments, a primer comprises a low self-complementarity score. A self-complementarity score is the likelihood that a tail will bind to itself. In some embodiments, the self-complementarity score is detected using Primer3Plus.

In some embodiments, a primer comprises a melting temperature of between about 50° C. and about 90° C., for example about 50° C., about 51° C., about 52° C., about 53° C., about 54° C., about 55° C., about 56° C., about 57, about 58, about 59, about 60, about 61, about 62, about 63, about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., or about 90° C.

In some embodiments, a primer comprises a fluorophore or a radioisotope. In some embodiments, the fluorophore is 6-FAM™, JOE™, TET™, Cal Fluor® Gold 540, HEX™, Cal Fluor Orange, TAMRA™, Cyanine 3, Quasar® 570, Cal Fluor Red 590, ROX™, Texas Red®, Cyanine 5, Quasar 670, Cyanine 5.5, or SYBR® Green I. In some embodiments, the radioisotope is selected from ³H, ²H, ¹⁴B, ¹⁴C, ²²Na, ²⁴Na, ²⁶Al, ³²Si, ³²P, ³⁷Ar, ⁴⁰K, ⁵¹Cr, ⁵⁴Mn, ⁵⁵Fe, ⁵⁷Co, ⁶⁰Co, ⁶⁶Ga, ⁶⁸Ga, ⁸⁵Kr, ⁸⁹Sr, ⁹⁰Sr, ⁹⁰Y, ^{99m}Tc, ¹⁰⁶Ru, ¹⁰⁶Rh, ¹¹²Ag, ¹⁰⁹Cd, ^{109m}Ag, ¹¹³n, ¹³²Te, ¹²⁵I, ¹²⁹I, ¹³¹I, ¹³³Xe, ³⁴Cs, 137Cs, ^{137m}Ba, ¹³³Ba, ¹⁴⁰La, ¹⁴⁴Ce, ¹⁴⁴Pr, ¹⁴⁴Nd, ¹⁵²Eu, ¹⁹²Ir, ¹⁹⁸Au, ²⁰⁴TI, ²⁰⁷Bi, ²²²Rn, ²¹⁸Po, ²¹⁴Pb ²¹⁴Bi, ²²⁶R, ²²⁸Th, ²³⁴U, ²³⁵U, ²³⁸U, ²³⁹Pu, ²⁴⁰Pu, ²⁴¹Am, ²⁵²Cf, ²⁵²Fm, or ²⁶⁸Mt.

### C. Cells

In some embodiments, the compositions and methods of the disclosure use cells. In some embodiments, the cells are competent cells. Competent cells are cells that take up nucleic acids like DNA. The competent cells utilized in the compositions and methods of the disclosure may be prokaryotic or eukaryotic cells. In some embodiments, the prokaryotic cells are bacteria, for example, genera of Escherichia, Klebsiella, Salmonella, Bacillus, Streptomyces, Streptococcus, Shigella, Staphylococcus, Corynebacterium, and Pseudomonas. In some embodiments, the eukaryotic cells are animal cells, for example, human cells or insect cells. In some embodiments, the eukaryotic cells are fungi or yeast. In some embodiments, the eukaryotic cells are filamentous fungal cells. In some embodiments, the filamentous fungal cells are protoplasts.

In some embodiments, the competent cells are provided in a concentration between about 1×10⁵ cells/mL and about 1×10¹⁰ cells/mL, for example, about 1×10⁵ cells/mL, 2×10⁵ cells/mL, 3×10⁵ cells/mL, 4×10⁵ cells/mL, 5×10⁵ cells/mL, 6×10⁵ cells/mL, 7×10⁵ cells/mL, 8×10⁵ cells/mL, 9×10⁵ cells/mL, 1×10⁶ cells/mL, 2×10⁶ cells/mL, 3×10⁶ cells/mL, 4×10⁶ cells/mL, 5×10⁶ cells/mL, 6×10⁶ cells/mL, 7×10⁶ cells/mL, 8×10⁶ cells/mL, 9×10⁶ cells/mL, 1×10⁷ cells/mL, 2×10⁷ cells/mL, 3×10⁷ cells/mL, 4×10⁷ cells/mL, 5×10⁷ cells/mL, 6×10⁷ cells/mL, 7×10⁷ cells/mL, 8×10⁷ cells/mL, 9×10⁷ cells/mL, 1×10⁸ cells/mL, 2×10⁸ cells/mL, 3×10⁸ cells/mL, 4×10⁸ cells/mL, 5×10⁸ cells/mL, 6×10⁸ cells/mL, 7×10⁸ cells/mL, 8×10⁸ cells/mL, 9×10⁸ cells/mL, 1×10⁹ cells/mL, 2×10⁹ cells/mL, 3×10⁹ cells/mL, 4×10⁹ cells/mL, 5×10⁹ cells/mL, 6×10⁹ cells/mL, 7×10⁹ cells/mL, 8×10⁹ cells/mL, 9×10⁹ cells/mL, or 1×10¹⁰ cells/mL.

### Filamentous Fungal Cells

In some embodiments, the competent cell is a filamentous fungal cell. Filamentous fungi form filamentous structures. In some embodiments, the filamentous fungal cell is used to prepare a protoplast. The filamentous fungus cell can be from any filamentous fungus strain known in the art or described herein including holomorphs, teleomorphs or anamorphs thereof. In some embodiments, the fungal cell can belong to either the Ascomycota or Basidiomycota phyla. Non-limiting examples of fungal strains include species of Achlya, Acremonium, Aspergillurs, Aureobasidium, Bjerkandera, Ceriporiopsis, Cephalosporium, Chrysosporium, Cochliobolus, Coriolus, Corynascus, Cryphonectria, Cryptococcus, Coprinus, Coriolus, Diplodia, Endothis, Filibasidium, Flumicola, Fusarium, Gibberella, Gliocladium, Humicola, Hypocrea, Magnaporthe. Myceliophthora (e.g., Myceliophthora thermophila), Mucor, Neocallimastix, Neurospora, Paecilomyces, Phanerochaete, Penicillium, Pleurotus, Podospora, Phlebia, Piromyces, Pyricularia, Rhizomucor, Rhizopus, Schizophyllur, Scytalidium, Sporotrichur, Talaromyces, Fhermoascus, Fhielavia, Thermoascus, Thielavia, Trametes, Tolypocladium, Trichoderma, Verticillium, Volvariella, or teleomorphs, anamorphs, synonyms, or taxonomic equivalents thereof. In some embodiments, the filamentous fungus is selected from the group consisting of A. nidulans, A. oryzae, A. sojae, and Aspergilli of the A. niger group. In some embodiments, the filamentous fungus is Aspergillus niger.

In some embodiments, mutants of the fungal species described herein are used in the compositions and methods of the disclosure. Examples of such mutants are strains that protoplast well; strains that produce primarily protoplasts with a single nucleus; strains that regenerate efficiently in microtiter plates, strains that regenerate faster and/or strains that take up polynucleotide (e.g., DNA) molecules efficiently, strains that produce cultures of low viscosity such as, for example, cells that produce hyphae in culture that are not so entangled as to prevent isolation of single clones and/or raise the viscosity of the culture, strains that have reduced random integration (e.g., disabled non-homologous end joining pathway) or combinations thereof.

In some embodiments, a mutant filamentous fungal strain lacks a selectable marker gene. In some embodiments, the mutant filamentous fungus strain is a uridine-requiring mutant strain. In some embodiments, the mutant strain is deficient in orotidine-5′-phosphate decarboxylase (OMPD), which is encoded by pyrG, or orotate p-ribosyl transferase (OPRT), which is encoded by pyrE. The following articles describe filamentous fungal strains and are incorporated by reference herein in their entirety: T. Goosen et al, Curr Genet. 1987, 11:499 503; J. Begueret et al., Gene. 1984 32:487 92.

In some embodiments, a mutant filamentous fungal strain possesses a compact cellular morphology characterized by shorter hyphae and a more yeast-like appearance. Examples of such mutants are filamentous fungal cells with altered gasl expression as described in U.S. Publication No. 2014/0220689, which is incorporated by reference herein in its entirety.

In some embodiments, a mutant filamentous fungal strain has an altered DNA repair system. In some embodiments, the altered DNA repair system is extremely efficient in homologous recombination and/or extremely inefficient in random integration. The efficiency of targeted integration of a genetic element of interest into the genome of the competent cell by homologous recombination, i.e. integration in a predetermined target locus, can be increased by augmented homologous recombination abilities and/or diminished non-homologous recombination abilities of the host cell. Augmentation of homologous recombination can be achieved by overexpressing one or more genes involved in homologous recombination (e.g., Rad51 and/or Rad52 protein). Removal, disruption or reduction in the activity of one or more non-homologous recombination pathways (e.g., the canonical non-homologous end joining (NHEJ) pathway, the Alternative NIHE or microhomology-mediated end-joining (Ait-NHEJ/MMEJ) pathway and/or the polymerase theta mediated end-joining (TMEJ) pathway) in the competent cells of the present disclosure can be achieved by any method known in that art such as, for example, by use of an antibody, a chemical inhibitor, a protein inhibitor, a physical inhibitor, a peptide inhibitor, or an anti-sense or RNAi molecule directed against a component of a specific non-homologous recombination (NHR) pathway (e.g., the NHEJ pathway, the Alt-NHEJ/MMEJ pathway and/or the TMEJ pathway).

In some embodiments, the activity of a single non-homologous end joining pathway is inhibited or reduced. In some embodiments, the activity of a combination of non-homologous end-joining pathways are inhibited or reduced such that the activity of one of the non-homologous end-joining pathways remains intact. In some embodiments, the activity of every non-homologous end-joining pathway is reduced or inhibited.

Examples of components of the NHEJ pathway that can be targeted for inhibition or reduction of activity alone or in combination can include, but are not limited to yeast KU70 or yeast KU80 or homologues or orthologs thereof. Examples of components of the Alt-NHEJ/MMEJ pathway that can be targeted for inhibition or a reduction in activity alone or in combination can include, but are not limited to a Polq gene, a Mre11 gene, an XPF-ERCCl gene or homologues or orthologs thereof. An example of a component of the NHEJ/MMEJ pathway that can be targeted for inhibition or a reduction in activity can include, but is not limited to a Polq gene or homologues or orthologs thereof. In some embodiments, the competent cell is deficient in one or more genes (e.g., yeast KU70, KU80 or homologues or orthologs thereof) of the NHEJ pathway. Examples of such mutants are cells with a deficient hdfA or hdfB gene as described in WO 05/95624. In some cases, a host-cell for use in the methods provided herein can be deficient in one or more genes of the Alternative NHEJ or microhomology-mediated end-joining (Alt-NHEJ/MMEJ) pathway and/or TMEJ pathway. Examples of such mutants are cells with that lack Polq gene or possess a mutant Polq gene as described in Wyatt et al. Essential roles for Polymerase 0 mediated end-joining in repair of chromosome breaks Mol Cell. 2016 Aug. 18; 63(4): 662-673.

In some embodiments, the methods and compositions described herein use fungal elements derived from filamentous fungi that may be readily separated from other such elements in a culture medium and are capable of reproducing. In some embodiments, the methods and compositions described herein use a fungal element selected from a spore, propagule, hyphal fragment, protoplast or micropellet.

### Production of Protoplasts

In some embodiments, the filamentous fungi cell is a protoplast. A protoplast is a fungal cell without a cell wall. In some embodiments, protoplasts are generated from filamentous fungi cells using the methods described herein or any known method in the art. Suitable procedures for preparation of protoplasts are known in the art including, for example, those described in EP 238,023 and Yelton et al. (1984, Proc. Natl. Acad. Sci. USA 81:1470-1474), which are incorporated by reference herein in their entirety.

In some embodiments, protoplasts are generated by treating a culture of filamentous fungal cells with one or more lytic enzymes or a mixture thereof. The lytic enzymes can be a beta-glucanase and/or a polygalacturonase. In some embodiments, the enzyme mixture for generating protoplasts is VINOTASTE (Novozymes A/S).

Following enzymatic treatment, the protoplasts can be isolated using methods known in the art. For example, undigested hyphal fragments can be removed by filtering the mixture through a porous barrier (such as Miracloth) in which the pores range in size from about 1 μm to about 200 μm, for example about 1 μm, about 2 μm, about 3 μm, about 4 μm, about 5 μm, about 6 μm, about 7 μm, about 8 μm, about 9 μm, about 10 μm, about 15 μm, about 20 μm, about 25 pm, about 30 μm, about 35 μm, about 40 μm, about 45 μm, about 50 μm, about 55 μm, about 60 pm, about 65 μm, about 70 μm, about 75 μm, about 80 μm, about 85 μm, about 90 μm, about 95 pm, about 100 μm, about 105 μm, about 110 μm, about 115 μm, about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 pm, about 195 μm, or about 200 μm in size.

In some embodiments, a filtrate containing protoplasts is centrifuged to cause the protoplasts to pellet to the bottom of the centrifuge tube. In some embodiments, a buffer of substantially lower osmotic strength is gently applied to the surface of the filtered protoplasts. The layered preparation can be centrifuged, which can cause the protoplasts to accumulate at a layer in the tube in which they are neutrally buoyant. Protoplasts can then be isolated from this layer for further processing. Following protoplast isolation, the remaining enzyme containing buffer can be removed by resuspending the protoplasts in an osmotic buffer and recollected by centrifugation. In some embodiments, the osmotic buffer is 1 M sorbitol buffered using tris(hydroxymethyl)aminomethane (TRIS). After sufficient removal of the enzyme containing buffer, the protoplasts can be resuspended in osmotically stabilized buffer also containing Calcium chloride. In some embodiments, protoplasts are resuspended to a final concentration between about 1×10⁵ protoplasts to about 1×10¹⁰ protoplasts per milliliter (mL). The pre-cultivation and the actual protoplasting step can be varied to optimize the number of protoplasts and the transformation efficiency. Any of the aforementioned steps may be repeated 1 time, 2 times, 3 times, 4 times, 5 times, 6 times, 7 times, 8 times, 9 times, 10 times, or more. Any of the aforementioned parameters may be varied. For example, there can be variations of inoculum size, inoculum method, pre-cultivation media, pre-cultivation times, pre-cultivation temperatures, mixing conditions, washing buffer composition, dilution ratios, buffer composition during lytic enzyme treatment, the type and/or concentration of lytic enzyme used, the time of incubation with lytic enzyme, the protoplast washing procedures and/or buffers, the concentration of protoplasts and/or polynucleotide and/or transformation reagents during the actual transformation, the physical parameters during the transformation, the procedures following the transformation up to the obtained transformants. Protoplasts can be resuspended in an osmotic stabilizing buffer. The composition of such buffers can vary depending on the species, application and needs. In some embodiments, the osmotic stabilizing buffer contains an organic component. Non-limiting examples of organic components include sucrose, citrate, mannitol, or sorbitol. In some embodiments, the osmotic stabilizing buffer contains an inorganic osmotic stabilizing component. Non-limiting examples of inorganic osmotic stabilizing components include KCl, buffers contain an inorganic osmotic stabilizing component like KCl, (NH₄)₂SO₄, MgSO₄, NaCl, or MgCl₂. Organic or inorganic components may be present in the osmotic stabilizing buffer between about 0.01 M and about 10 M, for example, about 0.01 M, about 0.02 M, about 0.03 M, about 0.04 M, about 0.05 M, about 0.06 M, about 0.07 M, about 0.08 M, about 0.09 M, about 0.1 M, about 0.2 M, about 0.3 M, about 0.4 M, about 0.5 M, about 0.6 M, about 0.7 M, about 0.8 M, about 0.9 M, about 1 M, about 1.1 M, about 1.2 M, about 1.3 M, about 1.4 M, about 1.5 M, about 1.6 M, about 1.7 M, about 1.8 M, about 1.9 M, about 2 M, about 2.1 M, about 2.2 M, about 2.3 M, about 2.4 M, about 2.5 M, about 2.6 M, about 2.7 M, about 2.8 M, about 2.9 M, or about 3 M.

In some embodiments, the osmotic stabilizing buffer is STC (sorbitol, calcium chloride, and TRIS, pH 8.0) or KCl-Citrate (KCl and citrate). In some embodiments, the protoplasts are used in a concentration between about 1×10⁵ cells/mL and about 1×10¹⁰ cells/mL, for example about 1×10⁵ cells/mL, 2×10⁵ cells/mL, 3×10⁵ cells/mL, 4×10⁵ cells/mL, 5×10⁵ cells/mL, 6×10⁵ cells/mL, 7×10⁵ cells/mL, 8×10⁵ cells/mL, 9×10⁵ cells/mL, 1×10⁶ cells/mL, 2×10⁶ cells/mL, 3×10⁶ cells/mL, 4×10⁶ cells/mL, 5×10⁶ cells/mL, 6×10⁶ cells/mL, 7×10⁶ cells/mL, 8×10⁶ cells/mL, 9×10⁶ ells/mL, 1×10⁷ cells/mL, 2×10⁷ cells/mL, 3×10⁷ cells/mL, 4×10⁷ cells/mL, 5×10⁷ cells/mL, 6×10⁷ cells/mL, 7×10⁷ cells/mL, 8×10⁷ cells/mL, 9×10⁷ cells/mL, 1×10⁸ cells/mL, 2×10⁸ cells/mL, 3×10⁸ cells/mL, 4×10⁸ cells/mL, 5×10⁸ cells/mL, 6×10⁸ cells/mL, 7×10⁸ cells/mL, 8×10⁸ cells/mL, 9×10⁸ cells/mL, 1×10⁸ cells/mL, 2×10⁸ cells/mL, 3×10⁹ cells/mL, 4×10⁹ cells/mL, 5×10⁹ cells/mL, 6×10⁹ cells/mL, 7×10⁹ cells/mL, 8×10⁹ cells/mL, 9×10⁹ cells/mL, or 1×10¹⁰ cells/mL. In some embodiments, the protoplasts are used in a concentration between about 1×10⁶ and about 1×10⁹ cells/mL. In some embodiments, the protoplasts are used in a concentration between about 1×10⁷ and about 5×10⁸ cells/mL. In some embodiments, the protoplasts are used in a concentration of 1×10⁸ cells/mL.

In some embodiments, after isolation of protoplasts, the protoplasts are cryopreserved. In some embodiments, the protoplasts are mixed with one or more cryoprotectants. The cryoprotectants can be glycols, dimethyl sulfoxide (DMSO), polyols, sugars, 2-Methyl-2,4-pentanediol (MPD), polyvinylpyrrolidone (PVP), methylcellulose, C-linked antifreeze glycoproteins (C-AFGP) or combinations thereof. Glycols for use as cryoprotectants in the methods and systems provided herein can be selected from ethylene glycol, propylene glycol, polypropylene glycol (PEG), glycerol, or combinations thereof. Polyols for use as cryoprotectants in the methods and systems provided herein can be selected from propane-1,2-diol, propane-1,3-diol, 1,1,1-tris-(hydroxymethyl)ethane (THME), and 2-ethyl-2-(hydroxymethyl)-propane-1,3-diol (EHMP), or combinations thereof. Sugars for use as cryoprotectants in the methods and systems provided herein can be selected from trehalose, sucrose, glucose, raffinose, dextrose or combinations thereof. In some embodiments, the protoplasts are mixed with DMSO. DMSO can be mixed with the protoplasts at a final concentration of at least, at most, less than, greater than, equal to, or about 1%, 2%, 3%, 4%, 5%, 6%, 7/a, 8%, 9%, 10%, 12.5%, 15%, 20/o, 25%, 30%, 35%, 40%, 45%, 50%, 55%, 60%, 65%, 70%, or 75% w/v or v/v. In some embodiments, the cryopreserved protoplasts are distributed to microtiter plates prior to storage. In some embodiments, the cryopreserved protoplasts are stored at a temperature from about −20° C. to about −80° C., for example about −20° C., about −22° C., about −24° C., about −26° C., about −28° C., about −30° C., about −32° C., about −34° C., about −36° C., about −38° C., about −40° C., about −42° C., about −44° C., about −46° C., about −48° C., about −50° C., about −52° C., about −54° C., about −56° C., about −58° C., about −60° C., about −62° C., about −64° C., about −66° C., about −68° C., about −70° C., about −72° C., about −74° C., about −76° C., about −78° C., or about −80° C.

In some embodiments, the protoplasts are stored for about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 1 week, about 2 weeks, about 3 weeks, about 4 weeks, about 5 weeks, about 6 weeks, about 1 month, about 2 months, about 3 months, about 4 months, about 5 months, about 6 months, about 7 months, about 8 months, about 9 months, about 10 months, about 11 months, about 1 year, about 18 months, about 2 years, about 3 years, about 4 years, about 5 years, about 6 years, about 7 years, about 8 years, about 9 years, about 10 years, or more.

### III. Methods for Detecting Ectopic Integration

Ectopic integration has significant and oftentimes deleterious consequences for individual genomes as a result of its effect on genome structure. Ectopic integration may lead to genomic deletions, inversions, translocations, and other rearrangements. The location of an ectopic integration is unpredictable, complicating the detection of such integrations. Current techniques, e.g., Southern Blot or Whole Genome Sequencing (WGS) to detect ectopic integration are time consuming and inefficient. Described herein is a superior strategy for detecting ectopic integration.

In some embodiments, the disclosure provides a method for detecting ectopic integration, comprising:
(a) transforming a cell with a genetic element, comprising a first tail, a second tail, a first homology arm, and a second homology arm; wherein the first tail is distal to the first homology arm and the second tail is distal to the second homology arm; and
(b) detecting ectopic integration of the genetic element by determining if a first tail or second tail is present in the genome of the cell;
wherein the presence of a first tail or second tail or both tails indicates ectopic integration.

In some embodiments, the method comprises transforming a cell with a genetic element. Any genetic element or cell described throughout this disclosure (for example, as described in Section II) may be utilized in the method for detecting ectopic integration.

In some embodiments, a genetic element comprises one nucleic acid. In some embodiments, a genetic element comprising one nucleic acid comprises from 5′ to 3′ a first tail, a first homology arm, a second homology arm, and a second tail. In some embodiments, the genetic element comprises a payload, selectable marker gene, or both. In some embodiments, a payload or selectable marker gene is located between the first homology arm and the second homology arm.

In some embodiments, a genetic element comprises two nucleic acids, the “first nucleic acid” and the “second nucleic acid.” In some embodiments, from 5′ to 3′, the first nucleic acid comprises a first tail and a first homology arm. In some embodiments, from 5′ to 3′, the second nucleic acid comprises a second homology arm and a second tail. In some embodiments, from 5′ to 3′, the first nucleic acid comprises a first tail, a first homology arm, and a second tail. In some embodiments, from 5′ to 3′, the second nucleic acid comprises a first tail, a second homology arm and a second tail. In some embodiments, the first nucleic acid, second nucleic acid, or both comprise a payload, or fragment thereof. In some embodiments, the first nucleic acid, second nucleic acid, or both comprise a selectable marker gene, or fragment thereof.

Tails and payloads of the genetic elements of this disclosure are described in Section II of this disclosure. In some embodiments, the nucleic acid sequence of the first tail or the second tail has a GC content between about 40% and about 60%. In some embodiments, the nucleic acid sequence of the first tail or the second tail comprises a primer binding site. In some embodiments, the nucleic acid sequence of the first tail or the second tail comprises a unique nucleic acid sequence that is not found in the genome of the transformed cell. In some embodiments, the nucleic acid sequence of the first tail or the second tail exhibits low-self complementarity.

In some embodiments, the cell is a prokaryotic cell. In some embodiments, the cell is a eukaryotic cell. In some embodiments, the cell is a fungal cell. In some embodiments, the cell is a filamentous fungal cell.

In some embodiments, the methods comprise transforming. Various methods for transformation are taught herein. In some embodiments, transformation of a competent cell involves heat-shock or electroporation. In some embodiments, transformation is automated. In some embodiments, competent cells are transformed using high-throughput electroporation systems, for example, the VWR®High-throughput Electroporation Systems, BTX™, Bio-Rad®Gene Pulser MXcell™, or other multi-well electroporation systems. In some embodiments, transformation is mediated by polyethylene glycol (PEG).

Transformation using the methods described herein can be facilitated through the use of any transformation reagent known in the art. Suitable transformation reagents can be selected from Polyethylene Glycol (PEG), FUGENE® HD (from Roche), Lipofectamine® or OLIGOFECTAMINE® (from Invitrogen), TRANSPASS®D1 (from New England Biolabs), LYPOVEC® or LIPOGEN® (from Invivogen). In some embodiments, PEG is the transformation/transfection reagent. PEG is available at different molecular weights and can be used at different concentrations.

In some embodiments, about 0.01 μg to about 100 μg of DNA, for example, about 0.01 μg, about 0.05 μg, about 0.1 μg, about 0.15 μg, about 0.2 μg, about 0.25 μg, about 0.3 μg, about 0.35 μg, about 0.4 μg, about 0.45 μg, about 0.5 μg, about 0.55 μg, about 0.6 μg, about 0.65 μg, about 0.7 μg, about 0.75 μg, about 0.8 μg, about 0.85 μg, about 0.9 μg, about 0.95 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, or about 100 μg of DNA (e.g. a genetic element) is used to transform a cell.

In some embodiments, the method comprises selecting for cells that comprise the genetic element (e.g., transformed competent cells). In some embodiments, cells that comprise the extra-chromosomally replicating plasmid are selected by applying a selective agent to the cells. Transformed competent cells grow in the presence of the selective agent as a result of a selectable marker gene whereas non-transformed competent cells which lack the selectable marker gene die in the presence of the selective agent.

Non-limiting examples of selective agents include antibiotics, such as ampicillin, tetracyclin, zeocin, spectinomycin, kanamycin, neomycin, vancomycin, methicillin, oxacillin, erythromycin, linezolid, puromycin, and hygromycin. Non-limiting examples of selectable marker genes include pyrG, hph, nat, andS, nptII, niaD, and argB.

In some embodiments, the selectable marker gene is an antibiotic resistance gene, for example, a chloramphenicol resistance gene, an ampicillin resistance gene, a tetracycline resistance gene, a Zeocin resistance gene, a spectinomycin resistance gene and a Km (Kanamycin resistance gene), tetA (tetracycline resistance gene), G418 (neomycin resistance gene), van (vancomycin resistance gene), methicillin (methicillin resistance gene), penicillin (penicillin resistance gene), oxacillin (oxacillin resistance gene), erythromycin (erythromycin resistance gene), linezolid (linezolid resistance gene), puromycin (puromycin resistance gene) or a hygromycin (hygromycin resistance gene).

In some embodiments, the methods comprise detecting ectopic integration of the genetic element by determining if a first tail or second tail is present in the genome of the cell. The presence of a tail is only detectable post-transformation if ectopic integration has occurred. Ectopic integration of a genetic element occurs via non-homologous end joining. Integration of a genetic element in the desired location of the genome of the target cell occurs via homologous recombination. Integration via homologous recombination results in loss of the tail sequences.

In some embodiments, detecting is performed using polymerase chain reaction (PCR). In some embodiments, the PCR reaction employs primers for detection of a first tail or a second tail. In some embodiments, the primers comprise fluorophores or radioisotopes, each of which are described in detail of Section II of this disclosure. In some embodiments, ectopic integration of a DNA fragment is confirmed using Southern Blotting or whole-genome sequencing.

### V. Methods for Determining the Quality of Genomic Transformation

In some embodiments, the disclosure provides methods for determining the quality of genomic transformation. In some embodiments, the method of determining the quality of genomic transformation in a cell population that is transformed with a genetic element comprising a first tail, a second tail, a first homology arm, a second homology arm, and a DNA fragment, comprises: (a) detecting ectopic integration of a DNA fragment by determining if a first tail or second tail is present in the genome of the cell, wherein the presence of a first tail or second tail indicates ectopic integration; and
(b) assigning the cell population a quality score, wherein a cell population that contains an ectopic integration has a lower quality score than a cell population without ectopic integration.

In some embodiments, detection of ectopic integration is performed using the compositions and according to the methods of Sections 11 or III of this disclosure.

In some embodiments, a cell population with on-target integration has a higher quality score than a cell population without on-target integration. In some embodiments, the cell population with on-target integration has a quality score that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 410%, at least 420%, at least 430%, at least 440%, at least 450%, at least 460%, at least 470%, at least 480%, at least 490%, or at least 500% higher than a cell population without on-target integration.

In some embodiments, a monoclonal cell population has a higher quality score than a polyclonal cell population. In some embodiments, the monoclonal cell population has a quality score that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%/o, at least 340%, at least 350%, at least 360%, at least 370%, at least 380%, at least 390%, at least 400%, at least 410%, at least 420%, at least 430%, at least 440%, at least 450%, at least 460%, at least 470%, at least 480%, at least 490%, or at least 500% higher than a polyclonal cell population.

In some embodiments, the method for determining the quality of genomic transformation, method for detecting ectopic integration, and compositions for detecting ectopic integration are used in conjunction with a HTP method of genomic engineering or techniques for programming genetic designs for implementation to host strains. Representative methods and techniques are described in U.S. Pat. Nos. 9,988,624, 10,336,998, 10,047,358, 10,457,933, and 10,647,980, each of which is incorporated by reference herein in its entirety.

### EXAMPLES

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1. Detecting Ectopic Integration after Transformation with a Genetic Element Comprising One Nucleic Acid

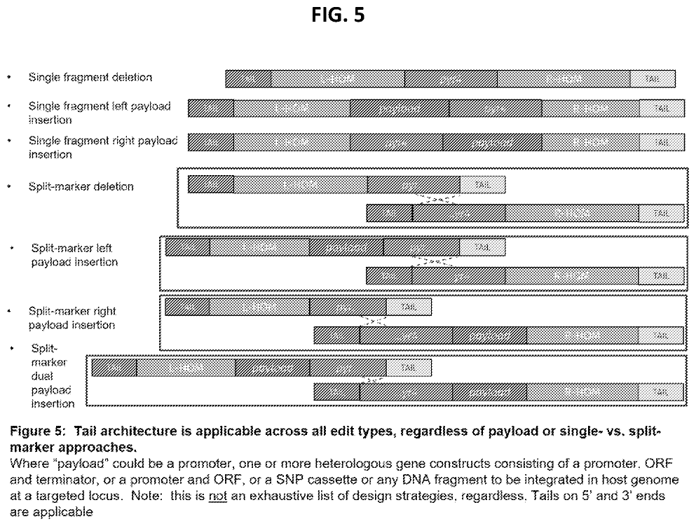

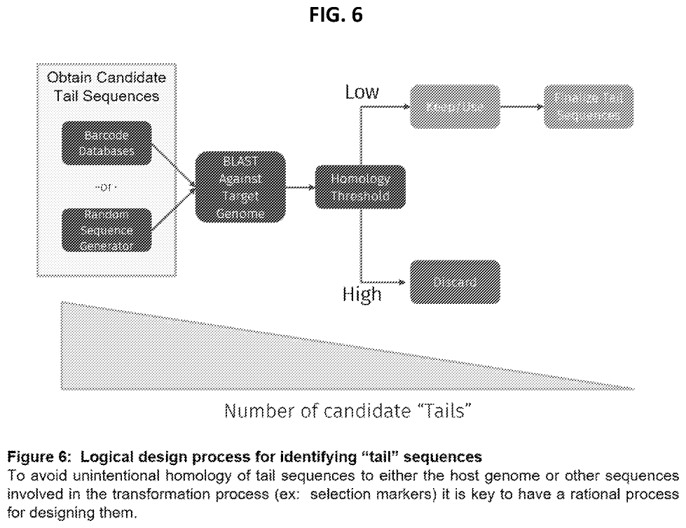

Filamentous fungal cells containing the gene genE are transformed with a nucleic acid comprising from 5′ to 3′ a first tail, a first homology arm, pyr4, a second homology arm, and a second tail (FIG. 5). On-target integration of pyr4 results in the replacement of genE with pyr4. The presence of ectopically integrated pyr4 is assessed by amplifying and detecting the presence of a first tail and/or a second tail. The presence of either tail indicates that pyr4 integrated ectopically into the genome.

### Example 2. Detecting Ectopic Integration after Transformation with a Genetic Element Comprising Two Nucleic Acids

Filamentous fungal cells containing the gene genE are transformed with two nucleic acids, a first nucleic acid and a second nucleic acid. The first nucleic acid comprises from 5′ to 3′ a first tail, a first homology arm, pyr4, and a second tail. The second nucleic acid comprises from 5′ to 3′ a first tail, pyr4, a second homology arm, and a second tail. The first nucleic acid contains the first portion of the split-marker pyr4 sequence starting from the 5′ end of pyr4, and the second nucleic acid contains the remaining portion of the split-marker pyr4 sequence such that between the first and second nucleic acids there is sufficient homology within pyr4 to undergo HR during genomic integration to complete the pyr4 open reading frame and such that each split part does not confer resistance or prototrophy. On-target integration of pyr4 results in the replacement of genE with pyr4. The presence of ectopically integrated pyr4 is assessed by amplifying and detecting the presence of a first tail and/or a second tail. The presence of either tail indicates that pyr4 integrated ectopically into the genome (FIG. 2).

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### ADDITIONAL EMBODIMENTS OF THE DISCLOSURE

The following embodiments are also envisioned by the present disclosure:

1. A method for detecting ectopic integration of a genetic element comprising:
(a) transforming a cell with a genetic element, comprising a first tail, a second tail, a first homology arm, and a second homology arm;
wherein the first tail is distal to the first homology arm and the second tail is distal to the second homology arm; and
(b) detecting ectopic integration of the genetic element by determining if a first tail or second tail is present in the genome of the cell;
wherein the presence of a first tail or second tail indicates ectopic integration.
   2. The method of embodiment 1, wherein the genetic element comprises two nucleic acids, wherein from 5′ to 3′, the first nucleic acid comprises the first tail, a first homology arm, and the second tail and wherein from 5′ to 3′, the second nucleic acid comprises a third tail, the second homology arm and a fourth tail.
   3. The method of embodiment 2, wherein the first tail and third tail have identical nucleic acid sequences.
   4. The method of embodiment 2, wherein the second tail and fourth tail have identical nucleic acid sequences.
   5. The method of embodiment 2, wherein the first tail and third tail have identical nucleic acid sequences, and wherein the second tail and the fourth tail have identical nucleic acid sequences.
   6. The method of any one of embodiments 2-5, wherein the presence of a third or a fourth tail in the genome of the cell indicates ectopic integration.
   7. The method of any one of embodiments 1-6, wherein 3′ of the first homology arm, the first nucleic acid comprises a fragment of a selectable marker gene.
   8. The method of any one of embodiments 2-7, wherein 5′ of the second homology arm, the second nucleic acid comprises a fragment of a selectable marker gene.
   9. The method of any one of embodiments 2-8, wherein 3′ of the first homology arm the first nucleic acid comprises a payload.
   10. The method of any one of embodiments 2-9, wherein 5′ of the second homology arm the second nucleic acid comprises a payload.
   11. The method of any one of embodiments 2-10, wherein the first nucleic acid and the second nucleic acid comprise a payload.
   12. The method of embodiment 1, wherein the genetic element comprises a nucleic acid, and wherein from 5′ to 3′, the nucleic acid comprises the first tail, the first homology arm, the second homology arm, and the second tail.
   13. The method of embodiment 12, wherein the nucleic acid comprises a selectable marker gene.
   14. The method of embodiment 12 or 13, wherein the nucleic acid comprises a payload.
   15. The method of embodiment 13 or 14, wherein the selectable marker gene is located between the first homology arm and the second homology arm.
   16. The method of embodiment 14 or 15, wherein the payload is located between the first homology arm and the second homology arm.
   17. The method of any one of embodiments 1-16, wherein the cell is a prokaryotic cell.
   18. The method of any one of embodiments 1-16, wherein the cell is a eukaryotic cell.
   19. The method of any one of embodiments 1-16, wherein the cell is a fungal cell.
   20. The method of any one of embodiments 1-19, wherein the first tail or the second tail comprises a unique nucleic acid sequence that is not found in the genome of the transformed cell.
   21. The method of any one of embodiments 1-20, wherein the nucleic acid sequence of the first tail or the second tail comprises a primer binding site.
   22. The method of any one of embodiments 1-21 wherein the nucleic acid sequence of the first tail or the second tail has GC content between 40% and 60%.
   23. The method of any one of embodiments 1-22, wherein the nucleic acid sequence of the first tail or the second tail exhibits low self-complementarity.
   24. The method of any one of embodiments 1-23, wherein the first tail or second tail are detected using a primer.
   25. The method of embodiment 24, wherein the primer comprises a fluorophore or a radioisotope.
   26. The method of any one of embodiments 1-25, wherein ectopic integration of a DNA fragment is confirmed using Southern Blotting or whole-genome sequencing.
   27. A composition for detecting ectopic integration, comprising:
(a) a genetic element, wherein the genetic element comprises a first tail, a second tail, a first homology arm, and a second homology arm;
wherein the first tail is distal to the first homology arm and the second tail is distal to the second homology arm;
(b) a primer that binds to the first tail; and
(c) a primer that binds to the second tail.
   28. The composition of embodiment 27, wherein the genetic element comprises two nucleic acids, wherein from 5′ to 3′, the first nucleic acid comprises the first tail, the first homology arm, and the second tail; and wherein from 5′ to 3′, the second nucleic acid comprises a third tail, the second homology arm and a fourth tail.
   29. The composition of embodiment 28, wherein the first tail and third tail have identical nucleic acid sequences.
   30. The composition of embodiment 28 or 29, wherein the second tail and fourth tail have identical nucleic acid sequences.
   31. The composition of any one of embodiments 28-30, wherein the first tail and third tail have identical nucleic acid sequences, and wherein the second tail and the fourth tail have identical nucleic acid sequences.
   32. The composition of any one of embodiments 28-31, wherein 3′ of the first homology arm, the first nucleic acid comprises a fragment of a selectable marker gene.
   33. The composition of any one of embodiments 28-32, wherein 5′ of the second homology arm, the second nucleic acid comprises a fragment of a selectable marker gene.
   34. The composition of any one of embodiments 28-33, wherein 3′ of the first homology arm the first nucleic acid comprises a payload.
   35. The composition of any one of embodiments 28-34, wherein 5′ of the second homology arm the second nucleic acid comprises a payload.
   36. The composition of any one of embodiments 28-35, wherein the first nucleic acid and the second nucleic acid comprise a payload.
   37. The composition of embodiment 27, wherein the genetic element comprises a nucleic acid, and wherein from 5′ to 3′, the nucleic acid comprises a first tail, a first homology arm, a second homology arm, and a second tail.
   38. The composition of embodiment 37, wherein the nucleic acid comprises a selectable marker gene.
   39. The composition of embodiment 37 or 38, wherein the nucleic acid comprises a payload.
   40. The composition of embodiment 37 or 38, wherein the selectable marker gene is located between the first homology arm and the second homology arm.
   41. The composition of embodiment 37 or 38, wherein the payload is located between the first homology arm and the second homology arm.
   42. The composition of any one of embodiments 27-41, wherein the nucleic acid sequence of the first tail or the second tail comprises a primer binding site.
   43. The composition of any one of embodiments 27-42, wherein the nucleic acid sequence of the first tail or the second tail has GC content between 40% and 60%.
   44. The composition of any one of embodiments 27-43, wherein the nucleic acid sequence of the first tail or the second tail exhibits low self-complementarity.
   45. A method of determining the quality of genomic transformation in a cell population that is transformed with a genetic element comprising a first tail, a second tail, a first homology arm, a second homology arm, and a DNA fragment, comprising:
(a) detecting ectopic integration of a DNA fragment by determining if a first tail or second tail is present in the genome of the cell, wherein the presence of a first tail or second tail indicates ectopic integration; and
(b) assigning the cell population a quality score, wherein a cell population that contains an ectopic integration has a lower quality score than a cell population without ectopic integration.
   46. The method of embodiment 45, wherein a cell population with on-target integration has a higher quality score than a cell population without on-target integration.
   47. The method of embodiment 45 or 46, wherein a monoclonal cell population has a higher quality score than a polyclonal cell population.

What is claimed is:

1. A method for detecting ectopic integration of a genetic element comprising:
(a) transforming a cell with a genetic element, comprising a first tail, a second tail, a first homology arm, and a second homology arm;
wherein the first tail is distal to the first homology arm and the second tail is distal to the second homology arm; and
(b) detecting ectopic integration of the genetic element by determining if a first tail or second tail is present in the genome of the cell;
wherein the presence of a first tail or second tail indicates ectopic integration.

2. The method of claim 1, wherein the genetic element comprises two nucleic acids, wherein from 5′ to 3′, the first nucleic acid comprises the first tail, a first homology arm, and the second tail and wherein from 5′ to 3′, the second nucleic acid comprises a third tail, the second homology arm and a fourth tail.

3. The method of claim 2, wherein the first tail and third tail have identical nucleic acid sequences.

4. The method of claim 2, wherein the second tail and fourth tail have identical nucleic acid sequences.

5. The method of claim 2, wherein the first tail and third tail have identical nucleic acid sequences, and wherein the second tail and the fourth tail have identical nucleic acid sequences.

6. The method of claim 2, wherein the presence of a third or a fourth tail in the genome of the cell indicates ectopic integration.

7. The method of claim 2, wherein 3′ of the first homology arm, the first nucleic acid comprises a fragment of a selectable marker gene.

8. The method of claim 2, wherein 5′ of the second homology arm, the second nucleic acid comprises a fragment of a selectable marker gene.

9. The method of claim 2, wherein 3′ of the first homology arm the first nucleic acid comprises a payload.

10. The method of claim 2, wherein 5′ of the second homology arm the second nucleic acid comprises a payload.

11. The method of claim 2, wherein the first nucleic acid and the second nucleic acid comprise a payload.

12. The method of claim 1, wherein the genetic element comprises a nucleic acid, and wherein from 5′ to 3′, the nucleic acid comprises the first tail, the first homology arm, the second homology arm, and the second tail.

13. The method of claim 12, wherein the nucleic acid comprises a selectable marker gene.

14. The method of claim 12, wherein the nucleic acid comprises a payload.

15. The method of claim 13, wherein the selectable marker gene is located between the first homology arm and the second homology arm.

16. The method of claim 14, wherein the payload is located between the first homology arm and the second homology arm.

17. The method of claim 1, wherein the cell is a prokaryotic cell.

18. The method of claim 1, wherein the cell is a eukaryotic cell.

19. The method of claim 1, wherein the cell is a fungal cell.

20. The method of claim 1, wherein the first tail or the second tail comprises a unique nucleic acid sequence that is not found in the genome of the transformed cell.

21. The method of claim 1, wherein the nucleic acid sequence of the first tail or the second tail comprises a primer binding site.

22. The method of claim 1, wherein the nucleic acid sequence of the first tail or the second tail has GC content between 40% and 60%.

23. The method of claim 1, wherein the nucleic acid sequence of the first tail or the second tail exhibits low self-complementarity.

24. The method of claim 1, wherein the first tail or second tail are detected using a primer.

25. The method of claim 24, wherein the primer comprises a fluorophore or a radioisotope.

26. The method of claim 1, wherein ectopic integration of a DNA fragment is confirmed using Southern Blotting or whole-genome sequencing.

27. A composition for detecting ectopic integration, comprising:
(a) a genetic element, wherein the genetic element comprises a first tail, a second tail, a first homology arm, and a second homology arm;
wherein the first tail is distal to the first homology arm and the second tail is distal to the second homology arm;
(b) a primer that binds to the first tail; and
(c) a primer that binds to the second tail.

28. The composition of claim 27, wherein the genetic element comprises two nucleic acids, wherein from 5′ to 3′, the first nucleic acid comprises the first tail, the first homology arm, and the second tail; and wherein from 5′ to 3′, the second nucleic acid comprises a third tail, the second homology arm and a fourth tail.

29. The composition of claim 28, wherein the first tail and third tail have identical nucleic acid sequences.

30. The composition of claim 28, wherein the second tail and fourth tail have identical nucleic acid sequences.

31. The composition of claim 28, wherein the first tail and third tail have identical nucleic acid sequences, and wherein the second tail and the fourth tail have identical nucleic acid sequences.

32. The composition of claim 28, wherein 3′ of the first homology arm, the first nucleic acid comprises a fragment of a selectable marker gene.

33. The composition of claim 28, wherein 5′ of the second homology arm, the second nucleic acid comprises a fragment of a selectable marker gene.

34. The composition of claim 28, wherein 3′ of the first homology arm the first nucleic acid comprises a payload.

35. The composition of claim 28, wherein 5′ of the second homology arm the second nucleic acid comprises a payload.

36. The composition of claim 28, wherein the first nucleic acid and the second nucleic acid comprise a payload.

37. The composition of claim 27, wherein the genetic element comprises a nucleic acid, and wherein from 5′ to 3′, the nucleic acid comprises a first tail, a first homology arm, a second homology arm, and a second tail.

38. The composition of claim 37, wherein the nucleic acid comprises a selectable marker gene.

39. The composition of claim 37, wherein the nucleic acid comprises a payload.

40. The composition of claim 37, wherein the selectable marker gene is located between the first homology arm and the second homology arm.

41. The composition of claim 37, wherein the payload is located between the first homology arm and the second homology arm.

42. The composition of claim 27, wherein the nucleic acid sequence of the first tail or the second tail comprises a primer binding site.

43. The composition of claim 27, wherein the nucleic acid sequence of the first tail or the second tail has GC content between 40% and 60%.

44. The composition of claim 27, wherein the nucleic acid sequence of the first tail or the second tail exhibits low self-complementarity.

45. A method of determining the quality of genomic transformation in a cell population that is transformed with a genetic element comprising a first tail, a second tail, a first homology arm, a second homology arm, and a DNA fragment, comprising:
(a) detecting ectopic integration of a DNA fragment by determining if a first tail or second tail is present in the genome of the cell, wherein the presence of a first tail or second tail indicates ectopic integration; and
(b) assigning the cell population a quality score, wherein a cell population that contains an ectopic integration has a lower quality score than a cell population without ectopic integration.

46. The method of claim 45, wherein a cell population with on-target integration has a higher quality score than a cell population without on-target integration.

47. The method of claim 45, wherein a monoclonal cell population has a higher quality score than a polyclonal cell population.
